(19) United States
(12) Patent Application Publication
(10) Pub. No.: US 2025/0195808 A1
(43) Pub. Date: Jun. 19, 2025

(54) NASAL MASK FOR SEALING THE AREA FROM THE NOSE TIP TO THE UPPER LIP

(71) Applicant: DCSTAR INC., New York, NY (US)

(72) Inventor: David LUO, New York, NY (US)

(21) Appl. No.: 18/540,315

(22) Filed: Dec. 14, 2023

(51) Int. Cl. A61M 16/06

(52) U.S. Cl. CPC A61M 16/0616; A61M 2210/0618

(57) ABSTRACT

A nasal mask for sealing the area from the nose tip to the upper lip of the user. It includes a flexible part that curves towards one side of the user's face. The flexible part has at least one protrusion, a chamber to accommodate gas, a first opening and at least one second opening. The flexible part includes a sealing section that, during use, surrounds the area at least including the nose tip, the first nostril, and the second nostril of the user, and makes contact with the area around the nostrils. The sealing section has at least one thin region and a support region that is thicker than the thin region. The nasal mask may also include a rigid part, an exhaust port, and a spring structure.

### TECHNICAL FIELD

This disclosure provides a nasal mask for sealing the area from the nose tip to the upper lip of the user, configured to encompass a part of the user's nose and to deliver pressurized gas from a ventilator into the user's nasal airway for the treatment of obstructive sleep breathing disorders. The nasal mask primarily includes a rigid part, a flexible part, a spring structure, and an exhaust port.

### BACKGROUND

Sleep is essential for everyone, serving as a fundamental aspect of life and a periodic physiological phenomenon. Nowadays, many people are in a sub-healthy state, frequently marked by fatigue. With the increasing prevalence of overweight and obese people as well as the growing issue of an aging population, more and more people are experiencing sleep disorders, making the incidence of sleep apnea a common problem worldwide, despite the fact that the incidence rates across different regions and populations are different. The most common type of sleep apnea is Obstructive Sleep Apnea Hypopnea Syndrome (OSAS). Recent studies have shown a notable increase in OSAS cases. From 2015 to 2019, the number of people aged 30 to 69 with OSAS rose from 980 million to 1.05 billion. Projections suggest that by 2024, about 1.15 billion people worldwide could be affected by OSAS, not including those below 30 or above 69 years of age. OSAS, also known as obstructive sleep apnea (OSA), is characterized by frequent episodes of apnea and hypoventilation while sleeping. Clinically, common symptoms of OSA include loud and irregular snoring, nocturnal choking sensations, disrupted sleep, excessive daytime sleepiness, memory decline, and, in severe cases, cognitive dysfunction and behavioral changes. The increasing prevalence of sleep apnea is evidently having a significant impact on the daily lives and well-being of people around the world.

Continuous Positive Airway Pressure (CPAP) therapy is a common treatment for OSAS, working by using positive pressure ventilation to keep the user's airway open, thereby reducing incidents of apnea and snoring. This treatment improves both sleep quality and overall quality of life for the user. The typical CPAP system includes components such as a ventilator, a hose, an elbow, a frame, and a patient interface device. Generally speaking, a CPAP treatment process involves generating a constant pressurized airflow from the CPAP machine, which is then delivered through a ventilator tube to a heated tube or a hose. Then the airflow passes through components like an elbow and a frame before reaching the patient interface device, which ultimately delivers the gas to at least one of airways of the user. In this process, the patient interface device is configured to make direct contact with the user's face, making it an essential component that must account for a range of human sensations. The design of the patient interface device is more intricate than that of other parts like the elbow and the frame, demanding not just compatibility between components and a precise, stable seal of the continuous positive pressure gas, but also the comfort for the user during facial contact.

Currently, there are broadly two types of patient interface devices on the market for sealing the user's nasal airway, catering to different preferences and needs: invasive nasal masks and non-invasive nasal masks. Invasive nasal masks are relatively smaller and have two small protrusions extending to one side. Typically made of silicone or rubber, the two protrusions of the air outlet are inserted into the nostrils during use, offering better sealing than non-invasive nasal masks. However, since part of the two protrusions enters the first nostril and the second nostril, some users may feel discomfort due to the foreign object sensation in their nostrils, making them less inclined to wear this type of sleep apnea treatment device. Meanwhile, invasive nasal masks also have higher requirements for the sealing structure and come with uncertainties that might not guarantee a secure seal. Therefore, for most users, invasive nasal masks may not be the best option.

Non-invasive nasal masks, which do not require a portion of the nasal mask to enter the user's nostrils, tend to be more comfortable for the user to use due to their reduced sensory impact. However, they also have their shortcomings when compared to invasive nasal masks. Non-invasive masks, which cover the outside of the user's nostrils, are bulkier than invasive masks, which can impact their convenience. Such masks are also heavier than invasive masks, potentially increasing the pressure felt on the nose during wear. Moreover, given the varying heights of different users' noses, existing non-invasive masks are not sized for such differences or do not offer flexible parts that can be adapted to varying heights. As mentioned above, both invasive and non-invasive nasal masks have their advantages and disadvantages, and to date, there is no comprehensive nasal mask on the market that combines the strengths of both while eliminating their weaknesses, thereby providing patients with OSAS with better therapeutic comfort and efficacy.

### SUMMARY

As such, this disclosure presents a nasal mask for sealing the area from the nose tip to the upper lip, combining the merits of both invasive and non-invasive nasal masks. This mask offers several advantages: it is more comfortable to wear, compact and easy to carry, lightweight, and exerts less pressure on the face. Additionally, it includes an adjustable spring structure tailored to accommodate varying heights of different users' noses, enhancing the nasal mask's adaptability and overall sealing effectiveness. Moreover, the nasal mask provided by this disclosure allows for compatibility with either a single air inlet or dual air inlets, thus providing users with more options.

This disclosure introduces a novel nasal mask for sealing the area from the nose tip to the upper lip of the user. Compared to existing non-invasive masks on the market, it offers a broader range of adaptability, better sealing, and a compact size for easier portability. The nasal mask in this disclosure surpasses invasive nasal masks in terms of comfort, exerting less pressure on the nostrils, thereby addressing the shortcomings of existing products. In this way, the disclosure has broader application scenarios and spaces, along with more accessible methods, offering a more manageable approach for continuous treatment of patients with OSAS.

A nasal mask for use under pressure conditions is provided herein, configured to deliver continuous positive pressure gas to a nasal airway of a user for treatment of sleep apnea, the nasal mask including at least some of the following elements or features.

A flexible part has a first opening near a face of the user and a second opening away from the face of the user. And the first opening, during use, is configured to surround an area at least including a nose tip, a first nostril, and a second nostril of the user during use, to make contact with the area around the nostrils.

The flexible part bends towards a side nearer the face of the user, forming at least one protrusion, and it includes at least one thin region and a support region thicker than the thin region, together forming a wall of the flexible part.

A rigid part includes at least two connection ports, in which one port is configured to connect to the second opening of the flexible part away from the face of the user and to seal with the flexible part, jointly forming a chamber to accommodate gas, and in which at least one port is configured to be an air inlet for positive pressure airflow to receive positive pressure airflow into the nasal mask.

In one embodiment, a wall thickness between the thin region and the support region of the sealing section changes uniformly.

In one embodiment, the first opening of the flexible part near the face of the user is larger than the second opening.

In one embodiment, the flexible part has a symmetrical structure.

In one embodiment, the rigid part has a wall of varying thicknesses.

In another embodiment, a nasal mask for use under pressure conditions is provided, configured to deliver continuous positive pressure gas to a nasal airway of a user for treatment of sleep apnea, the nasal mask including at least some of the following elements or features.

A flexible part bends towards a side nearer a face of the user, forming at least one protrusion and creating a chamber for accommodating gas, which includes a first opening near the face of the user and at least one second opening away from the face of the user. The second opening is configured to be an air inlet to receive continuous positive pressure airflow into the flexible part.

The flexible part includes a sealing section, configured to be a wall formed by a protrusion extending at the first opening, which surrounds an area at least including a nose tip, a first nostril, and a second nostril of the user during use, and which makes contact with the area around the nostrils.

The sealing section includes at least one thin region and a support region thicker than the thin region, together forming a wall of the sealing section.

In one embodiment, the nasal mask includes a rigid part that connects to the second opening of the flexible part away from the face of the user, with the rigid part being configured to form a connection with other components for the treatment of sleep disorders.

In one embodiment, the support region of the sealing section has more than one wall thickness.

In one embodiment, the wall thickness between the thin region and the support region of the sealing section changes uniformly.

In one embodiment, the area of the support region of the sealing section is larger than the area of the thin region of the sealing section.

In yet another embodiment, a nasal mask for use under pressure conditions is provided, configured to deliver continuous positive pressure gas to a nasal airway of a user for treatment of sleep apnea, the nasal mask including at least some of the following elements or features.

A flexible part bends towards a side nearer a face of the user, forming at least one protrusion and creating a chamber for accommodating gas, which includes a first opening near the face of the user and at least one second opening away from the face of the user. The second opening is configured to be an air inlet for continuous positive pressure airflow to enter the flexible part.

The flexible part includes a sealing section, configured to be a wall formed by the protrusion extending at the first opening, which surrounds an area at least including a nose tip, a first nostril, and a second nostril of the user during use, and which makes contact with the area around the nostrils.

An exhaust port includes at least one hole, for discharging positive pressure gas from the chamber through the hole to an external environment.

In one embodiment, the nasal mask includes a rigid part that connects to the second opening of the flexible part away from the face of the user, configured to form a connection with other components for the treatment of sleep disorders.

In one embodiment, the exhaust port is configured to be detachably joined with the flexible part.

In one embodiment, the sealing section has more than one wall thickness.

In one embodiment, the first opening of the flexible part near the face of the user is larger than the second opening.

In another embodiment, a nasal mask for use under pressure conditions is provided, configured to deliver continuous positive pressure gas to a nasal airway of a user for treatment of sleep apnea, the nasal mask including at least some of the following elements or features.

A flexible part bends towards a side nearer to a face of the user, forming at least one protrusion and creating a chamber for accommodating gas, which includes a first opening near the face of the user and at least one second opening away from the face of the user. The second opening is configured to be an air inlet for continuous positive pressure airflow to enter the flexible part.

The flexible part includes a main body of the flexible part and a sealing section configured to be a wall formed by the protrusion extending at the first opening, which surrounds an area at least including a nose tip, a first nostril, and a second nostril of the user during use, and which makes contact with the area around the nostrils.

The sealing section includes at least one thin region and a support region thicker than the thin region, together forming a wall of the sealing section.

A spring structure with collapsible walls is provided between the main body of the flexible part and the sealing section, wherein the spring structure has at least one of the following features:
1) the height of the spring structure is at or between 5 to 30 mm;
2) the width of the spring structure is at or between 3 to 40 mm;
3) the wall thickness of the spring structure is less than 3 mm.

In one embodiment, the nasal mask includes a rigid part that connects to the second opening of the flexible part away from the face of the user, with the rigid part being configured to form a connection with other components for the treatment of sleep disorders.

In one embodiment, an average wall thickness of the main body of the flexible part is greater than the average wall thickness of the sealing section.

In one embodiment, the sealing section is configured to seal with the face of the user without positioning or contacting a columella of the user.

In one embodiment, the spring structure has a different wall thickness than the main body of the flexible part and the sealing section.

In yet another embodiment, a nasal mask for use under pressure conditions is provided, configured to deliver continuous positive pressure gas to a nasal airway of a user for treatment of sleep apnea, the nasal mask including at least some of the following elements or features.

A flexible part includes a first opening near a face of the user and at least one second opening away from the face of the user, configured to surround an area at least including a nose tip, a first nostril, and a second nostril of the user during use, to make contact with the area around the nostrils.

The flexible part bends towards a side of the face of the user, forms at least one protrusion and creates a chamber for accommodating gas. The flexible part has at least one thin region and a support region thicker than the thin region, together forming a wall of the flexible part.

A comfort layer is configured to attach to at least a portion of the flexible part and to join with the face of the user.

In one embodiment, a material of the comfort layer includes a sponge or a fabric. In one embodiment, the first opening of the flexible part near the face of the user is larger than the second opening.

In one embodiment, an inner edge of the comfort layer is greater than or equal to an edge of the first opening of the flexible part. During use, the comfort layer and the flexible part jointly define a pathway for an airflow to enter the nasal airway in a sealed manner.

In one embodiment, the inner edge of the comfort layer is smaller than the edge of the first opening of the flexible part, and during use, the comfort layer alone defines the pathway for the airflow to enter the nasal airway in a sealed manner.

The benefits of a nasal mask for sealing the area from the nose tip to the upper lip of the user provided by this disclosure can at least include:

1) Compared to existing non-invasive nasal masks on the market, which seal the area from below the nose tip to the upper lip, the nasal mask in this disclosure offers better sealing. Since there are always variations in the size and height of users' noses, existing non-invasive nasal masks often fail to accommodate such differences. They typically seal the face of the user using two openings that contact the area around the nostrils, to deliver continuous positive pressure airflow. However, due to variations in nasal height and size, the openings in existing nasal masks may not adapt to most users' nasal airways properly, leading to issues like gas leakage. As such, this disclosure introduces a nasal mask that seals the area from the nose tip to the upper lip. This nasal mask includes a portion of the nose tip in its seal. A spring structure is provided at the first opening that delivers continuous positive pressure airflow and comes into contact with the nose tip. The spring structure helps to adjust to fit different users' noses and resolves the issues of poor sealing and potential gas leakage common in non-invasive nasal masks compared to invasive ones. It ensures an airtight fit without the risk of gas leakage due to a mismatch with the user's nose. This design not only achieves better sealing but also reduces discomfort or foreign body sensation inside the nostrils, enhancing user comfort, reducing treatment interruptions or non-compliance with treatment plans due to user discomfort.

2) Compared to existing nasal masks on the market that seal the area from the nose bridge to the upper lip, the nasal mask in this disclosure is smaller in volume, lighter in weight, and more convenient. This nasal mask adjusts the sealing range to cover from the nose tip to the upper lip area, reducing its volume compared to similar existing nasal masks. A nasal mask with a smaller volume is easier for users to wear and easier to adjust when the mask shifts, reducing the potential risk of gas leakage to some extent. Due to its smaller volume, the nasal mask of this disclosure is more user-friendly for carrying and storing during travel, facilitating continuous treatment. It also increases wearing comfort by reducing the contact area with the user's face, making activities like reading easier while wearing the nasal mask.

3) Compared to invasive nasal masks, the nasal mask in this disclosure is more comfortable, imposes less burden on the nostrils, and is more inclusive. This is because the air outlet of the nasal mask does not enter the first nostril and the second nostril openings of the user, thereby reducing sensory interference and making it more suitable for a broader range of users, especially for those who are not accustomed to wearing sleep apnea treatment devices, so that such people can be more easily adapted to wearing the treatment device. Invasive nasal masks often struggle to fit the nostril sizes of most users. In contrast, the non-invasive nasal mask in this disclosure only needs to ensure that the first opening encompasses the user's nasal airway, resulting in greater comfort, less burden on the nostrils, and increased inclusivity with less sensory disturbance, aiding users in falling asleep more easily.

4) The air inlet of the nasal mask in this disclosure can be configured in different forms. Users have the option to choose between dual air inlets on both sides or a single air inlet in the middle, offering a wide range of choices and adaptability. This simplifies the product, reducing production and research and development costs. The nasal mask in this disclosure offers flexibility in the design of the air inlet for the positive pressure airflow. The flexible part of the same design that contacts the user's face can be adapted to fit different joint parts with either a single air inlet or two air inlets on both sides. This extends the technical features of existing products, allowing users to choose or replace parts based on their needs or preferences, thus providing them with more options. In this way, this design of the nasal mask can adapt to new demands and changes on the market. Besides, independently developing and testing the flexible part that contacts the user's face, as opposed to designing and developing the entire respiratory accessory, reduces the complexity and risk of the product, leading to simpler and more cost-effective production methods. Consequently, this disclosure also reduces the research and development costs of the product.

### BRIEF DESCRIPTION OF THE DRAWINGS

FIG. 1 is a three-dimensional schematic diagram of a nasal mask for sealing the area from the nose tip to the upper lip of the user in accordance with an embodiment;

FIG. 2 is a usage scenario diagram of a nasal mask for sealing the area from the nose tip to the upper lip of the user in accordance with an embodiment;

FIG. 3 is a diagram showing the sealing range of a nasal mask for sealing the area from the nose tip to the upper lip of the user in accordance with an embodiment;

FIG. 4 is a diagram of a nasal mask for sealing the area from the nose tip to the upper lip of the user and its contact with the area around the nostrils in accordance with an embodiment;

FIG. 5 is an exploded diagram of a nasal mask for sealing the area from the nose tip to the upper lip of the user in accordance with an embodiment;

FIG. 6 is a front view and a cross-sectional structural diagram along line A-A of a nasal mask for sealing the area from the nose tip to the upper lip of the user in accordance with an embodiment;

FIG. 7 is a front view and a cross-sectional structural diagram along line B-B of a nasal mask for sealing the area from the nose tip to the upper lip of the user in accordance with an embodiment;

FIG. 8 is a schematic diagram showing the symmetrical structure of the flexible part in a nasal mask for sealing the area from the nose tip to the upper lip of the user in accordance with an embodiment;

FIG. 9 is a cross-sectional diagram of the sealing section of a nasal mask for sealing the area from the nose tip to the upper lip of the user, showing different wall thicknesses in accordance with an embodiment;

FIG. 10 is a cross-sectional diagram of another form of a nasal mask for sealing the area from the nose tip to the upper lip of the user, showing different wall thicknesses in accordance with an embodiment;

FIG. 11 is a three-dimensional schematic diagram of another form of a nasal mask with a single air inlet for sealing the area from the nose tip to the upper lip of the user in accordance with an embodiment;

FIG. 12 is a three-dimensional schematic diagram of a form of a nasal mask with a rigid part for sealing the area from the nose tip to the upper lip of the user in accordance with an embodiment;

FIG. 13 is a three-dimensional schematic diagram of another form of a nasal mask with a rigid part for sealing the area from the nose tip to the upper lip of the user in accordance with an embodiment;

FIG. 14 is a three-dimensional schematic diagram of a nasal mask with an exhaust port for sealing the area from the nose tip to the upper lip of the user in accordance with an embodiment;

FIG. 15 is a three-dimensional schematic diagram of another form of a nasal mask with an exhaust port for sealing the area from the nose tip to the upper lip of the user in accordance with an embodiment;

FIG. 16 is a diagram showing the positional relationship between the first opening and the exhaust port of the patient interface device in accordance with Embodiment 3;

FIG. 17 is a three-dimensional schematic diagram of a nasal mask with a spring structure for sealing the area from the nose tip to the upper lip of the user in accordance with an embodiment;

FIG. 18 is a cross-sectional diagram of a nasal mask for sealing the area from the nose tip to the upper lip of the user with a spring structure being squeezed in accordance with an embodiment;

FIG. 19 is a three-dimensional schematic diagram of a nasal mask with a comfort layer for sealing the area from the nose tip to the upper lip of the user in accordance with an embodiment;

FIG. 20 is a three-dimensional schematic diagram of a nasal mask with a comfort layer made of another material for sealing the area from the nose tip to the upper lip of the user in accordance with an embodiment;

FIG. 21 is a three-dimensional schematic diagram of a nasal mask with different forms of the comfort layer for sealing the area from the nose tip to the upper lip of the user in accordance with an embodiment.

### DETAILED DESCRIPTION

To facilitate the understanding of the disclosure, a more comprehensive description will be provided with reference to the related drawings. The drawings illustrate typical embodiments of the disclosure. However, the disclosure can be implemented in many different forms and is not limited to the embodiments described herein. On the contrary, these embodiments are provided to make the disclosure more thorough and comprehensive.

Unless otherwise defined, all technical and scientific terms used herein have the same meaning as commonly understood by one of ordinary skill in the art to which this disclosure belongs. The terms used in this specification are for the purpose of describing particular embodiments only rather than limiting the disclosure.

The disclosure acknowledges the constraints in adaptability and the pronounced discomfort users experience with existing invasive nasal masks on the market. Likewise, it notes the drawbacks of current non-invasive nasal masks, such as their overly large volume and lack of sealing stability. As such, the disclosure herein provides a nasal mask 1 for sealing the area from the nose tip to the upper lip of the user, and a spring structure 23 is provided in the flexible part 2, which allows the nasal mask 1 to be adjusted according to the user's nose, thereby improving the stability of the seal. Since the first opening 12 that contacts the user's face is configured to envelop the nasal airway, the nasal mask 1 accommodates a wide range of nose heights and sizes. The nasal mask 1 has a smaller volume, making it convenient for the user to carry for continuous treatment and timely adjustment in case of shifts. This novel design enhances the product's flexibility and has a positive impact on patients with OSAS.

Detailed embodiments are presented below to elucidate several structures of a nasal mask for sealing the area from the nose tip to the upper lip of the user.

### Embodiment 1

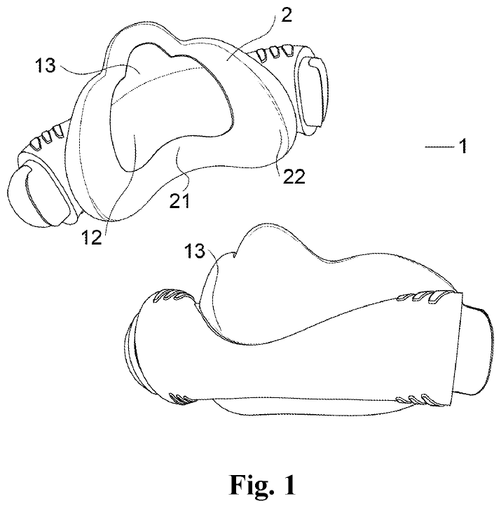

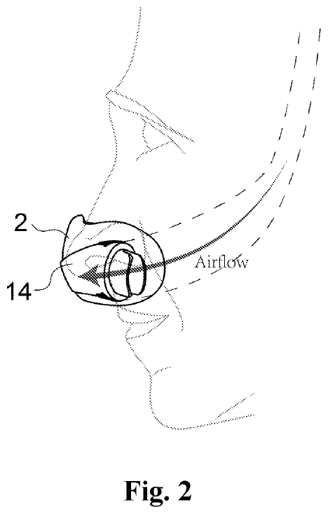

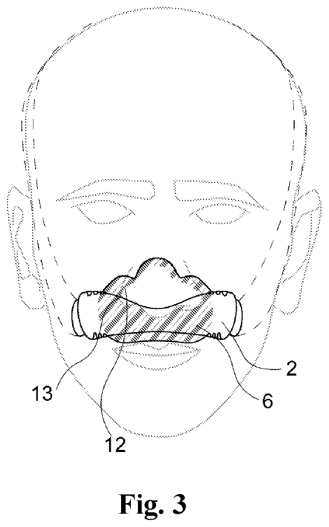

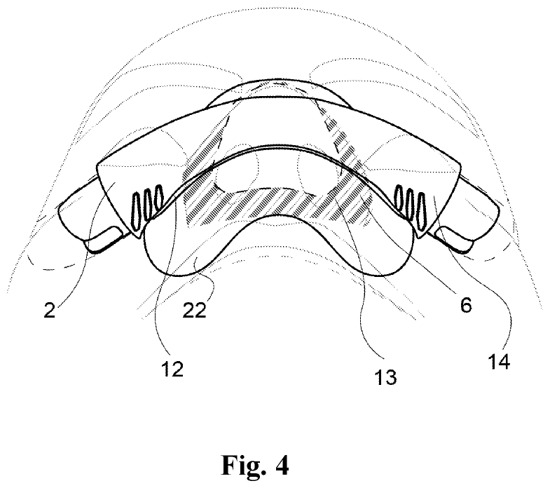

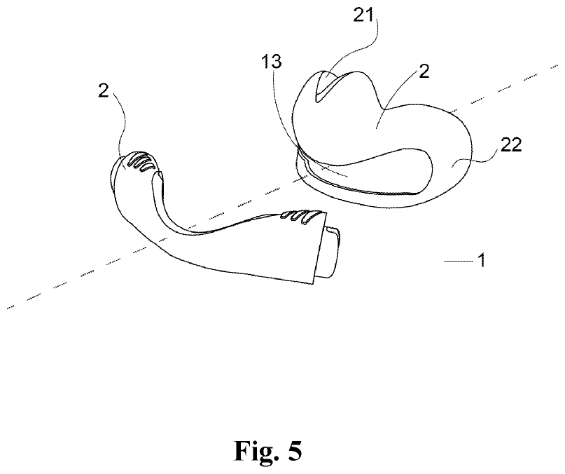

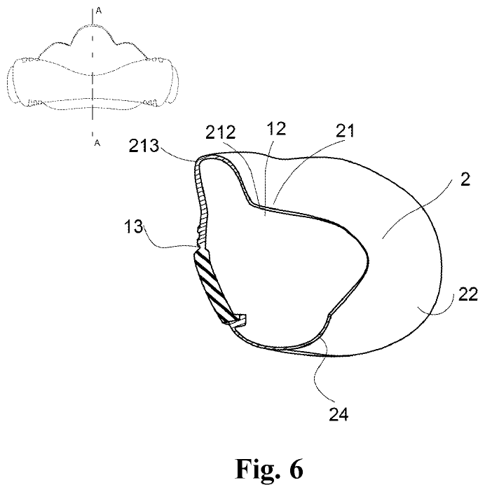

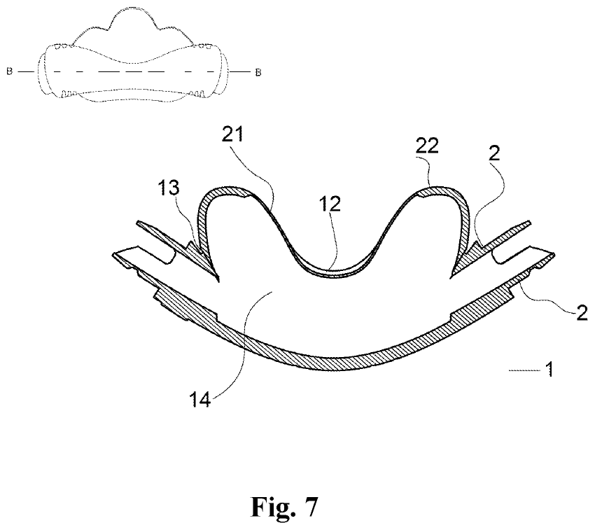

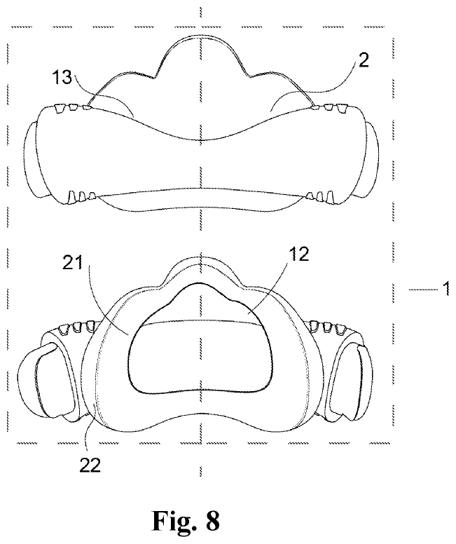

the upper lip of the user, which is for use under pressure conditions and is configured to deliver continuous positive pressure gas to the user's nasal airway for the treatment of sleep apnea. This embodiment includes a three-dimensional schematic diagram, a usage scenario diagram, a diagram showing the sealing range, an exploded diagram, and cross-sectional structural diagrams, as shown in FIGS. 1-11. The nasal mask 1 presented in this embodiment includes a flexible part 2 that bends towards the side nearer to the user's face, forming at least one protrusion 22 and creating a chamber 14 to accommodate gas, a first opening near the user's face and at least one second opening 12 away from the user's face, with the second opening 13 being configured to be an air inlet to receive continuous positive pressure airway into the flexible part 2. The flexible part 2 includes a main body and a sealing section 21, where the average wall thickness of the main body is greater than that of the sealing section 21. The sealing section 21 is configured to be a wall formed by the protrusion 22 extending at the first opening 12, which surrounds an area at least including the nose tip, the first nostril, and the second nostril of the user during use, which makes contact with the area around the nostrils (as shown in FIGS. 3 and 4). This design has clear advantages in the existing market, as it falls somewhere between a nasal mask that seals the area from the nose bridge to the upper lip and one that seals only the area from below the nose tip to the upper lip. The nasal mask 1 in this disclosure encompasses the user's nose tip, thus avoiding compatibility issues stemming from variations in nose height and size across users. It is crafted to ensure that the opening that contacts the user's face sufficiently encircles the nasal airway, thereby eliminating concerns over excessive or insufficient size or misalignment with the user's nostrils, which could result in slippage. Distinct from existing non-invasive nasal masks that seal the area from below the nose tip to the upper lip, the nasal mask 1 seals the area from the user's nose tip to the upper lip and includes a sealing section 21 configured to seal with the face of the user without positioning or contacting the columella of the user (as shown in FIG. 4). This configuration reduces the contact area of the sealing section 21 with the user's face, enhancing the user's quick adjustment to wearing the nasal mask 1. The non-invasive method, compared to invasive nasal masks, reduces the foreign body sensation when using the nasal mask 1, thereby amplifying comfort, consequently, increasing patient compliance with the treatment.

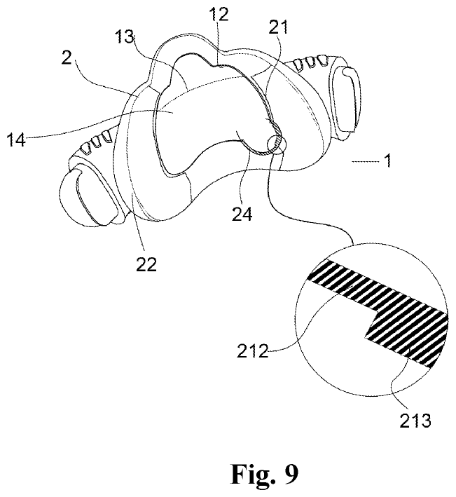

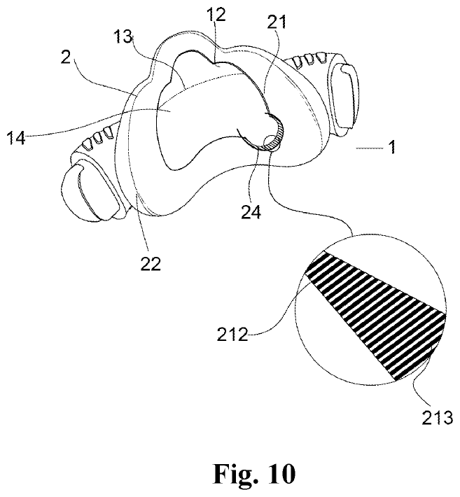

The sealing section 21 of the nasal mask 1 has more than one thickness and it includes at least one thin region 212 and a support region 213 that is thicker than the thin region 212. The wall thickness of the thin region 212 is at or between 0.2 to 1.2 mm. The thin region 212 and the support region 213 together form the wall of the sealing section 21. During use, the sealing section 21 of the flexible part 2 contacts the user's face to form a seal. To ensure that the sealing section 21 provides adequate facial support while remaining comfortable for the user, a part of the wall at the sealing section 21 is thickened to form the support region 213, with the remaining part as the thin region 212 (as shown in FIG. 9). In some forms of the nasal mask 1, the area of the support region 213 in the sealing section 21 is greater than that of the thin region 212, providing effective and stable support for the sealing section 21 of the nasal mask 1. In some forms of nasal mask 1 in Embodiment 1, to maintain the aesthetics of the sealing section 21, the wall thickness can be configured to change uniformly, transitioning from a thicker wall to the thin region 212. This type of the support region 213 of the sealing section 21 has more than one wall thickness (as shown in FIG. 10). Therefore, the flexible part 2 has at least one thin region and a support region 213 that is thicker than the thin region, together forming the wall of the flexible part 2, and in some embodiments, the flexible part 2 has a symmetrical structure.

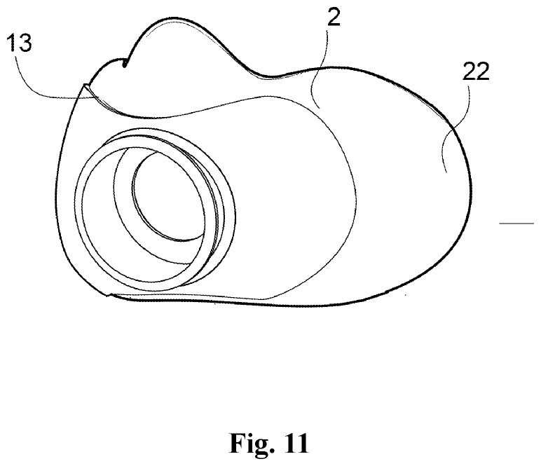

In another embodiment, the nasal mask 1 includes an air inlet. This nasal mask 1 can be used in conjunction with components like an elbow and a frame, configured to receive continuous positive pressure airflow into the nasal mask 1 (as shown in FIG. 11).

### Embodiment 2

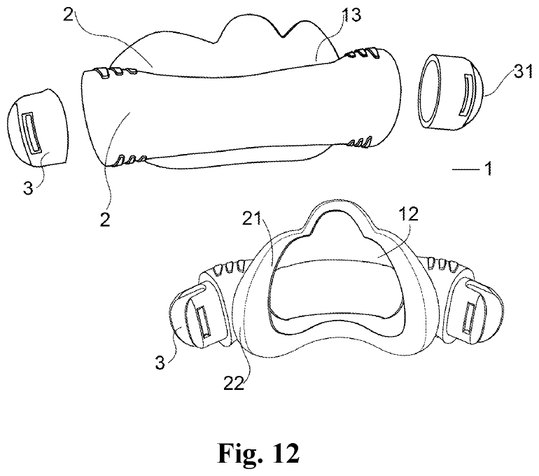

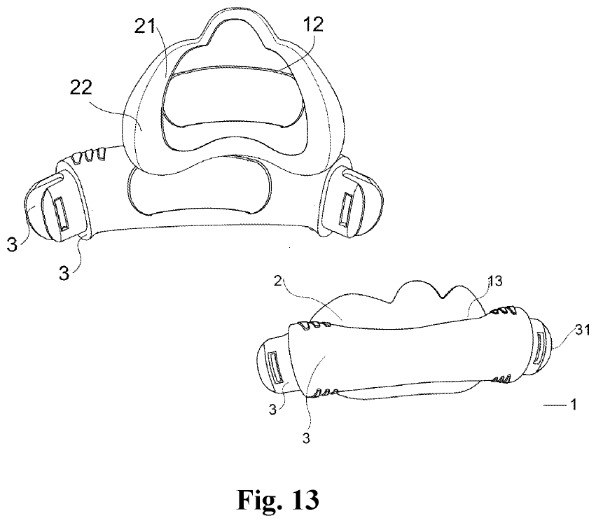

the upper lip of the user, as illustrated in FIGS. 12-13. This embodiment offers a three-dimensional schematic diagram of the patient interface device. In the embodiment of this disclosure shown in FIGS. 12-13, what differentiates this nasal mask 1 from that in Embodiment 1 is the inclusion of a rigid part 3. The rigid part 3, connected to the second opening 13 of the flexible part 2, which is away from the user's face, is configured to form a connection with other sleep disorder treatment components. The rigid part 3 has at least two connection ports 31, one of which is configured to connect to the second opening 13 of the flexible part 2, sealing with the flexible part 2 to jointly form a chamber 14 to accommodate gas. At least one of the connection ports 31 is configured to be an air inlet for the positive pressure airflow, receiving the positive pressure airflow into the nasal mask 1. The rigid part 3 comes in two forms. One form includes two separate parts placed on the nasal mask 1 at the air inlet for receiving continuous pressurized airflow, serving as connection ports 31 for inter-component connections. Since the connection ports 31 of the rigid part 3 generally connect to other components through a snap-fit structure, in this case, the rigid part 3 has a wall of varying thicknesses for a snap-fit connection (as illustrated in FIG. 12). The other form is that the rigid part 3, as a single unit, seals and connects with the second opening 13 of the flexible part 2, which is away from the user's face. In this form, the rigid part 3 not only includes connection ports 31 for connecting with other components but also has a certain internal space connected with the flexible part 2 for forming a chamber 14 to accommodate gas (as shown in FIG. 13). In this case, the first opening 12 of the flexible part 2, near the user's face, is larger than the second opening 13 that is the opening for connecting the rigid part 3 and the flexible part 2. When the nasal mask 1 receives gas, the gas enters through the connection port 31 of the rigid part 3, follows the path along the inner wall of the rigid part 3, and then proceeds through the flexible part 2 into the user's nasal airway.

### Embodiment 3

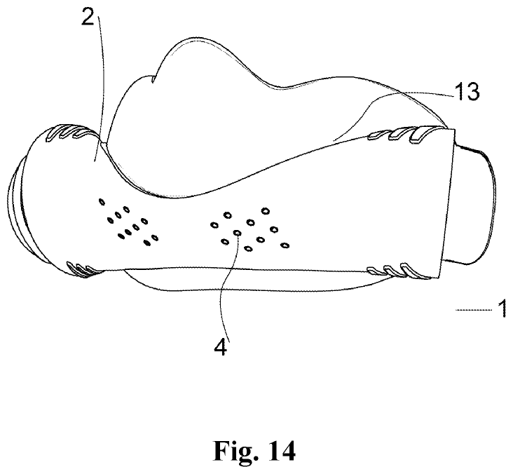

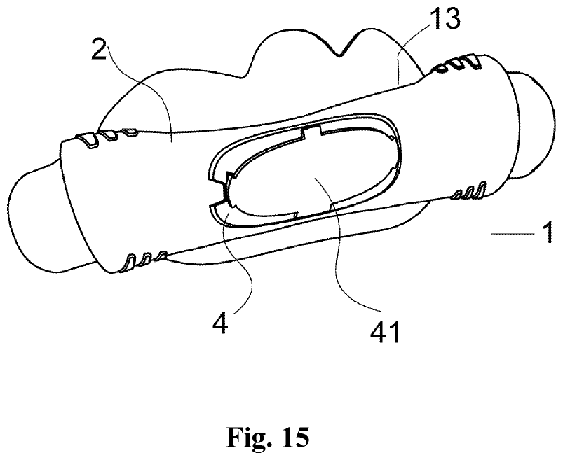

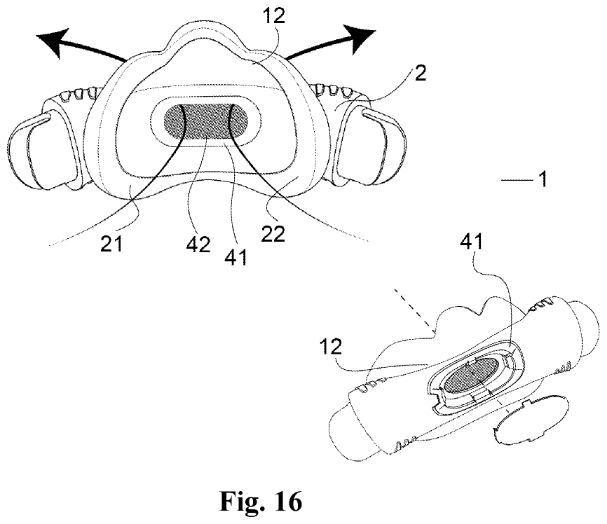

the upper lip of the user, as shown in FIGS. 14-16. It offers a three-dimensional schematic diagram of the patient interface device and a diagram showing the positional relationship between the first opening 12 and the exhaust port 4. In the embodiment of this disclosure depicted in FIGS. 14-16, the difference from the nasal mask 1 in Embodiment 1 is the inclusion of an exhaust port 4. The exhaust port 4 has at least one hole, configured to discharge the positive pressure gas from the chamber 14 through the hole to the external environment. The hole is positioned opposite the opening (i.e., the first opening 12) on the nasal mask 1 that directly contacts and seals with the user's face (as shown in FIG. 16). The exhaust port 4 is configured to be detachably joined with the flexible part 2. When the user wears the nasal mask 1 for exhausting, the gas first flows from the user's airway to the sealing section 21. Since the exhaust port 4 is opposite the sealing section 21, the waste gas can be directly expelled to the external environment in an almost straight line through the exhaust port 4. This configuration can take two forms. One form involves small holes drilled directly opposite the opening (the first opening 12) on the nasal mask 1 that directly contacts and seals with the user's face, spaced a certain distance apart. These holes can have a certain taper to disperse the airflow about to be expelled, reducing noise to some extent; this form is relatively simple (as depicted in FIG. 14). The other form involves a larger hole provided opposite the opening (the first opening 12) on the nasal mask 1 that directly contacts and seals with the user's face, configured for mounting an external exhaust component 41. The larger hole can connect to the external exhaust component 41 through a clamping structure, a friction fit, an adhesive bond, thermal pressing, or other methods. The external exhaust component 41 can include a noise reduction material 42. When the user wears the nasal mask 1 for exhausting, the gas first flows from the user's airway to the sealing section 21, then is expelled to the external environment through the external exhaust component 41. The noise reduction effect of the nasal mask 1 is achieved by the noise reduction material 42 within the external exhaust component 41, which could be one or more of a fiber-type, foam-type, or granular-type material (as shown in FIG. 15).

### Embodiment 4

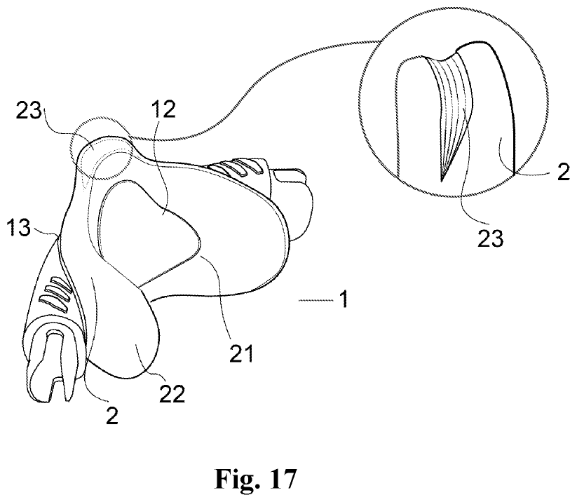

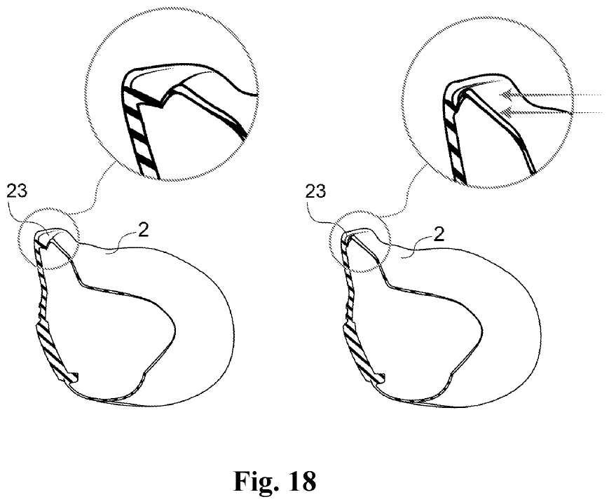

This embodiment provides a nasal mask 1 for sealing the area from the nose tip to the upper lip of the user, as shown in FIGS. 17-18. It offers a three-dimensional schematic diagram of the patient interface device. In this embodiment of the disclosure, as depicted in FIGS. 17-18, the difference from the nasal mask 1 in Embodiment 1 lies in the inclusion of a spring structure 23 with collapsible walls, provided between the main body of the flexible part and the sealing section 21. The spring structure 23 has at least one of the following features: 1) the height of the spring structure is at or between 5 to 30 mm; 2) the width of the spring structure is at or between 3 to 40 mm; 3) the wall thickness of the spring structure is less than 3 mm. The flexible part 2 includes a first opening 12 near the user's face and a second opening 13 away from the user's face. The first opening 12 is configured to surround the area at least including a nose tip, a first nostril, and a second nostril of the user during use, to make contact with the area around the nostrils. The spring structure 23 has a different wall thickness than the main body of the flexible part and the sealing section 21. The sealing section 21 cannot be a completely soft wall and requires a wall of a certain thickness to maintain the shape of the support region 213 and to ensure a seal with the user's face. The main body of the flexible part primarily forms a chamber 14 to accommodate gas. The wall of the spring structure 23 cannot be too thick to facilitate easy folding of the spring structure 23, hence the wall thickness of the spring structure 23 should be thinner than that of the sealing section 21 and the main body of the flexible part. Given the differences in the size and height of users' noses, the presence of the spring structure 23 in the nasal mask 1 serves a dual purpose. On one hand, it enables the nasal mask 1 to adapt to users' noses of varying heights and sizes, addressing the issue of existing nasal masks where the two openings do not completely fit most users' nasal airways, often leading to gas leakage. On the other hand, since the nose tip is typically higher than other areas, the collapsible walls of the spring structure 23 provide a cushioning effect at the nose tip when wearing the nasal mask 1. The design of the spring structure 23 helps to reduce the pressure exerted by the sealing section 21 at the nose tip, thereby reducing discomfort caused by excessive pressure on the nose tip during use.

### Embodiment 5

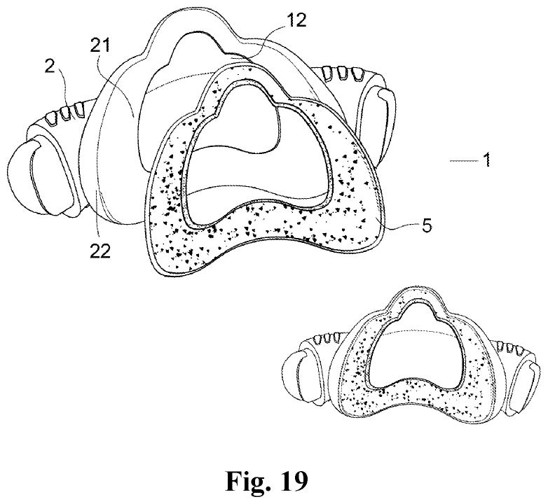

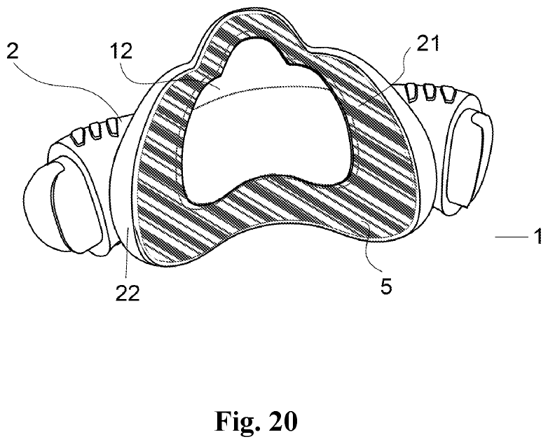

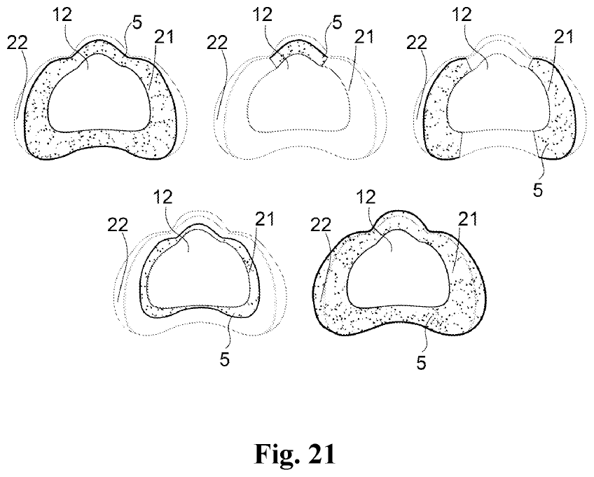

This embodiment provides a nasal mask 1 for sealing the area from the nose tip to the upper lip of the user, as depicted in FIGS. 19-21. It includes a three-dimensional schematic diagram of the patient interface device. The difference of this nasal mask 1 from that in Embodiment 1, as shown in FIGS. 19-21, is the inclusion of a comfort layer 5. This comfort layer 5 is configured to attach to at least a portion of the flexible part 2 and to join with the user's face. The use of the comfort layer 5 makes the nasal mask 1 both durable and comfortable, since the part of the nasal mask 1 that contacts the face is breathable, preventing skin symptoms like red marks that may result from prolonged wear, thus enhancing user compliance with treatment. The material of the comfort layer 5 can include a sponge or a fabric, and its connection to the sealing section 21 can be direct or indirect. Direct connection methods include silicone adhesive, thermal pressing, molding, etc. There are two ways for the comfort layer 5 to connect to the sealing section 21 of the nasal mask 1. Specifically, in one way, the inner edge of the comfort layer 5 is greater than or equal to the edge of the first opening 12 of the flexible part 2. During use, the comfort layer 5 and the flexible part 2 jointly define a pathway for airflow to enter the user's nasal airway in a sealed manner. In the other way, the inner edge of the comfort layer 5 is smaller than the edge of the first opening 12 of the flexible part 2, with the comfort layer 5 alone defining the pathway for the airflow to enter the nasal airway in a sealed manner. The connection between the nasal mask 1 and the comfort layer 5 includes, but is not limited to, adhesives, thermal melting, welding, and other detachable or non-detachable connections.

Furthermore, it is possible to combine the technical features of the above embodiments as needed to obtain a nasal mask for sealing the area from the nose tip to the upper lip of the user that includes all or some of the aforementioned technical features.

The benefits of a nasal mask for sealing the area from the nose tip to the upper lip of the user provided by this disclosure can at least include:

1) Compared to existing non-invasive nasal masks on the market, which seal the area from below the nose tip to the upper lip, the nasal mask in this disclosure offers better sealing. Since there are always variations in the size and height of users' noses, existing non-invasive nasal masks often fail to accommodate such differences. They typically seal the face of the user using two openings that contact the area around the nostrils, to deliver continuous positive pressure airflow. However, due to variations in nasal height and size, the openings in existing nasal masks may not adapt to most users' nasal airways properly, leading to issues like gas leakage. As such, this disclosure introduces a nasal mask that seals the area from the nose tip to the upper lip. This nasal mask includes a portion of the nose tip in its seal. A spring structure is provided at the first opening that delivers continuous positive pressure airflow and comes into contact with the nose tip. The spring structure helps to adjust to fit different users' noses and resolves the issues of poor sealing and potential gas leakage common in non-invasive nasal masks compared to invasive ones. It ensures an airtight fit without the risk of gas leakage due to a mismatch with the user's nose. This design not only achieves better sealing but also reduces discomfort or foreign body sensation inside the nostrils, enhancing user comfort, reducing treatment interruptions or non-compliance with treatment plans due to user discomfort.

2) Compared to existing nasal masks on the market that seal the area from the nose bridge to the upper lip, the nasal mask in this disclosure is smaller in volume, lighter in weight, and more convenient. This nasal mask adjusts the sealing range to cover from the nose tip to the upper lip area, reducing its volume compared to similar existing nasal masks. A nasal mask with a smaller volume is easier for users to wear and easier to adjust when the mask shifts, reducing the potential risk of gas leakage to some extent. Due to its smaller volume, the nasal mask of this disclosure is more user-friendly for carrying and storing during travel, facilitating continuous treatment. It also increases wearing comfort by reducing the contact area with the user's face, making activities like reading easier while wearing the nasal mask.

3) Compared to invasive nasal masks, the nasal mask in this disclosure is more comfortable, imposes less burden on the nostrils, and is more inclusive. This is because the air outlet of the nasal mask does not enter the first nostril and the second nostril openings of the user, thereby reducing sensory interference and making it more suitable for a broader range of users, especially for those who are not accustomed to wearing sleep apnea treatment devices, so that such people can be more easily adapted to wearing the treatment device. Invasive nasal masks often struggle to fit the nostril sizes of most users. In contrast, the non-invasive nasal mask in this disclosure only needs to ensure that the first opening encompasses the user's nasal airway, resulting in greater comfort, less burden on the nostrils, and increased inclusivity with less sensory disturbance, aiding users in falling asleep more easily.

4) The air inlet of the nasal mask in this disclosure can be configured in different forms. Users have the option to choose between dual air inlets on both sides or a single air inlet in the middle, offering a wide range of choices and adaptability. This simplifies the product, reducing production and research and development costs. The nasal mask in this disclosure offers flexibility in the design of the air inlet for the positive pressure airflow. The flexible part of the same design that contacts the user's face can be adapted to fit different joint parts with either a single air inlet or two air inlets on both sides. This extends the technical features of existing products, allowing users to choose or replace parts based on their needs or preferences, thus providing them with more options. In this way, this design of the nasal mask can adapt to new demands and changes on the market. Besides, independently developing and testing the flexible part that contacts the user's face, as opposed to designing and developing the entire respiratory accessory, reduces the complexity and risk of the product, leading to simpler and more cost-effective production methods. Consequently, this disclosure also reduces the research and development costs of the product.

Several embodiments of the disclosure have been described in conjunction with the accompanying drawings. It should be noted that the disclosure is not limited to the specific embodiments mentioned above. These particular embodiments are merely illustrative, not restrictive. Those of ordinary skill in this field, in light of the teachings of the disclosure and without departing from the spirit and scope of the disclosure as claimed, can make various modifications and variations. All such modifications and variations are within the protection scope of the disclosure.

It must be noted that as used herein and in the appended claims, the singular forms “a”, “an”, and “the” include their plural equivalents, unless the context clearly dictates otherwise.

1. A nasal mask for use under pressure conditions, configured to deliver continuous positive pressure gas to a nasal airway of a user for treatment of sleep apnea, the nasal mask comprising:
a flexible part with a first opening configured to be near a face of the user and a second opening configured to be away from the face of the user,
wherein the first opening, during use, is configured to surround only an area including a nose tip, a first nostril, and a second nostril of the user, and to make contact with an area around the first nostril and the second nostril on the face of the user and a nose of the user,
wherein the flexible part is configured to bend towards a side nearer the face of the user, to form at least one protrusion,
wherein the flexible part includes at least one thin region forming a sealing section and a support region that is thicker than the thin region, together to form a wall of the flexible part;
wherein the nasal mask further comprises a rigid part with at least two connection ports, wherein one port is configured to connect to the second opening of the flexible part that is configured to be away from the face of the user and to seal with the flexible part, to jointly form a chamber to accommodate gas,
wherein at least one port is configured to be an air inlet for positive pressure airflow to receive positive pressure airflow into the nasal mask, and
wherein the sealing section is configured to seal with the face of the user and the nose of the user and enclose the nose tip without contacting a columella and the nose tip of the user.

2. The nasal mask according to claim 1, wherein the first opening of the flexible part that is configured to be near the face of the user is larger than the second opening.

3. The nasal mask according to claim 1, wherein the flexible part has a symmetrical structure.

4. The nasal mask according to claim 1, wherein the rigid part has a wall of varying thicknesses.

5. A nasal mask for use under pressure conditions, configured to deliver continuous positive pressure gas to a nasal airway of a user for treatment of sleep apnea, the nasal mask comprising:
a flexible part that is configured to bend towards a side nearer a face of the user, is configured to form at least one protrusion and to create a chamber to accommodate gas, and includes a first opening that is configured to be near the face of the user and at least one second opening that is configured to be away from the face of the user,
wherein the second opening is configured to be an air inlet to receive continuous positive pressure airflow into the flexible part;
wherein the flexible part includes a sealing section that is configured to be a wall formed by a protrusion extending at the first opening, which is configured to surround only an area including a nose tip, a first nostril, and a second nostril of the user during use, and which makes contact with an area around the first nostril and the second nostril on the face of the user and a nose of the user,
wherein the sealing section includes at least one thin region and a support region that is thicker than the thin region, together to form a wall of the sealing section, and
wherein the sealing section is configured to seal with the face of the user and the nose of the user and enclose the nose tip without contacting a columella and the nose tip of the user.

6. The nasal mask according to claim 5, wherein the nasal mask includes a rigid part that connects to the at least one second opening of the flexible part that is configured to be away from the face of the user, with the rigid part being configured to form a connection with other components for treatment of sleep disorders.

7. The nasal mask according to claim 5, wherein the support region of the sealing section has more than one wall thickness.

8. The nasal mask according to claim 5, wherein a wall thickness between the thin region and the support region of the sealing section changes uniformly.

9. The nasal mask according to claim 5, wherein an area of the support region of the sealing section is larger than an area of the thin region of the sealing section.

10. A nasal mask for use under pressure conditions, configured to deliver continuous positive pressure gas to a nasal airway of a user for treatment of sleep apnea, the nasal mask comprising:
a flexible part that is configured to bend towards a side nearer a face of the user, is configured to form at least one protrusion and to create a chamber to accommodate gas, and includes a first opening configured to be near the face of the user and at least one second opening configured to be away from the face of the user,
wherein the second opening is configured to be an air inlet for continuous positive pressure airflow to enter the flexible part;
wherein the flexible part includes a sealing section, configured to be a wall formed by the protrusion extending at the first opening, which is configured to surround only an area at least including a nose tip, a first nostril, and a second nostril of the user during use, and which makes contact with an area around the first nostril and the second nostril on the face of the user and a nose of the user; and
an exhaust port, with at least one hole to discharge positive pressure gas from the chamber through the hole to an external environment,
wherein the sealing section is configured to seal with the face of the user and the nose of the user and enclose the nose tip without contacting a columella and the nose tip of the user.

11. The nasal mask according to claim 10, wherein the nasal mask includes a rigid part that connects to the at least one second opening of the flexible part that is configured to be away from the face of the user, with the rigid part being configured to form a connection with other components for treatment of sleep disorders.

12. The nasal mask according to claim 10, wherein the exhaust port is configured to be detachably joined with the flexible part.

13. The nasal mask according to claim 10, wherein the sealing section has more than one wall thickness.

14. The nasal mask according to claim 10, wherein the first opening of the flexible part that is configured to be near the face of the user is larger than the second opening.

15. (canceled)

16. A nasal mask for use under pressure conditions, configured to deliver continuous positive pressure gas to a nasal airway of a user for treatment of sleep apnea, the nasal mask comprising:
a flexible part, with a first opening configured to be near a face of the user and at least one second opening configured to be away from the face of the user, wherein the first opening is configured to surround only an area including a nose tip, a first nostril, and a second nostril of the user during use, and to make contact with an area around the first nostril and the second nostril on the face of the user and a nose of the user, and
wherein the flexible part is configured to bend towards a side of the face of the user, configured to form at least one protrusion and to create a chamber to accommodate gas,
wherein the flexible part has at least one thin region forming a sealing section and a support region that is thicker than the thin region, together to form a wall of the flexible part,
wherein the sealing section is configured to seal with the face of the user and the nose of the user and enclose the nose tip without contacting a columella and the nose tip of the user; and
the nasal mask further comprising
a comfort layer, configured to attach to at least a portion of the flexible part and to join with the face of the user.

17. The nasal mask according to claim 16, wherein a material of the comfort layer includes a sponge or a fabric.

18. The nasal mask according to claim 16, wherein the first opening of the flexible part that is configured to be near the face of the user is larger than the second opening.

19. The nasal mask according to claim 16, wherein an inner edge of the comfort layer is greater than or equal to an edge of the first opening of the flexible part, and wherein during use, the comfort layer and the flexible part jointly define a pathway for an airflow to enter the nasal airway in a sealed manner.

20. The nasal mask according to claim 19, wherein the inner edge of the comfort layer is smaller than the edge of the first opening of the flexible part, and wherein during use, the comfort layer alone defines the pathway for the airflow to enter the nasal airway in a sealed manner.

21. The nasal mask according to claim 1, further comprising a spring structure provided between a main body of the flexible part and the sealing section, wherein the spring structure is configured to adjust to different user's noses.
